SEQUENCE LISTING (30 sequences)

SEQ ID NO: 1 - protein, 7 aa, Artificial Sequence
  SYNTHESIZED
PKKKRKV

SEQ ID NO: 2 - protein, 7 aa, Artificial Sequence
  SYNTHESIZED
PKKKRRV

SEQ ID NO: 3 - protein, 16 aa, Artificial Sequence
  SYNTHESIZED
KRPAATKKAGQAKKKK

SEQ ID NO: 4 - protein, 11 aa, Artificial Sequence
  SYNTHESIZED
YGRKKRRQRRR

SEQ ID NO: 5 - protein, 9 aa, Artificial Sequence
  SYNTHESIZED
RKKRRQRRR

SEQ ID NO: 6 - protein, 9 aa, Artificial Sequence
  SYNTHESIZED
PAAKRVKLD

SEQ ID NO: 7 - protein, 11 aa, Artificial Sequence
  SYNTHESIZED
RQRRNELKRSP

SEQ ID NO: 8 - protein, 8 aa, Artificial Sequence
  SYNTHESIZED
VSRKRPRP

SEQ ID NO: 9 - protein, 8 aa, Artificial Sequence
  SYNTHESIZED
PPKKARED

SEQ ID NO: 10 - protein, 8 aa, Artificial Sequence
  SYNTHESIZED
PQPKKKPL

SEQ ID NO: 11 - protein, 12 aa, Artificial Sequence
  SYNTHESIZED
SALIKKKKKMAP

SEQ ID NO: 12 - protein, 7 aa, Artificial Sequence
  SYNTHESIZED
PKQKKRK

SEQ ID NO: 13 - protein, 10 aa, Artificial Sequence
  SYNTHESIZED
RKLKKKIKKL

SEQ ID NO: 14 - protein, 10 aa, Artificial Sequence
  SYNTHESIZED
REKKKFLKRR

SEQ ID NO: 15 - protein, 20 aa, Artificial Sequence
  SYNTHESIZED
KRKGDEVDGVDEVAKKKSKK

SEQ ID NO: 16 - protein, 17 aa, Artificial Sequence
  SYNTHESIZED
RKCLQAGMNLEARKTKK

SEQ ID NO: 17 - protein, 38 aa, Artificial Sequence
  SYNTHESIZED
NQSSNFGPMKGGNFGGRSSGPYGGGGQYFAKPRNQGGY

SEQ ID NO: 18 - protein, 42 aa, Artificial Sequence
  SYNTHESIZED
RMRIZFKNKGKDTAELRRRRVEVSVELRKAKKDEQILKRRNV

SEQ ID NO: 19 - protein, 20 aa, Artificial Sequence
  SYNTHESIZED
GRKKRRQRRRPPQPKKKRKV

SEQ ID NO: 20 - protein, 19 aa, Artificial Sequence
  SYNTHESIZED
PLSSIFSRIGDPPKKKRKV

SEQ ID NO: 21 - protein, 24 aa, Artificial Sequence
  SYNTHESIZED
GALFLGWLGAAGSTMGAPKKKRKV

SEQ ID NO: 22 - protein, 27 aa, Artificial Sequence
  SYNTHESIZED
GALFLGFLGAAGSTMGAWSQPKKKRKV

SEQ ID NO: 23 - protein, 21 aa, Artificial Sequence
  SYNTHESIZED
KETWWETWWTEWSQPKKKRKV

SEQ ID NO: 24 - protein, 11 aa, Artificial Sequence
  SYNTHESIZED
YARAAARQARA

SEQ ID NO: 25 - protein, 11 aa, Artificial Sequence
  SYNTHESIZED
THRLPRRRRRR

SEQ ID NO: 26 - protein, 11 aa, Artificial Sequence
  SYNTHESIZED
GGRRARRRRRR

SEQ ID NO: 27 - protein, 12 aa, Artificial Sequence
  SYNTHESIZED
RRQRRTSKLMKR

SEQ ID NO: 28 - protein, 27 aa, Artificial Sequence
  SYNTHESIZED
GWTLNSAGYLLGKINLKALAALAKKIL

SEQ ID NO: 29 - protein, 33 aa, Artificial Sequence
  SYNTHESIZED
KALAWEAKLAKALAKALAKHLAKALAKALKCEA

SEQ ID NO: 30 - protein, 16 aa, Artificial Sequence
  SYNTHESIZED
RQIKIWFQNRRMKWKK

(19) United States
(12) Patent Application Publication
(10) Pub. No.: US 2021/0238222 A1
(43) Pub. Date: Aug. 5, 2021

(54) PRODUCING RECOMBINANT PROTEINS WITH REDUCED LEVELS OF HOST CELL PROTEINS

(71) Applicant: Sigma-Aldrich Co. LLC, St. Louis, MO (US)

(72) Inventors: Trissa Borgschulte, St. Charles, MO (US); Joaquina Mascarenhas, St. Louis, MO (US); Martin Zillmann, Shewsbury, MA (US); Frank Swartzwelder, St. Louis, MO (US); Kevin Kayser, St. Louis, MO (US)

(21) Appl. No.: 17/052,624

(22) PCT Filed: May 3, 2019
(86) PCT No.: PCT/US19/30609

§ 371 Date: Nov. 3, 2020

Related U.S. Application Data
(60) Provisional application No. 62/667,198, filed on May 4, 2018.

(51) Int. Cl. C07K 1/13; C07K 1/22; C07K 1/36; C12N 15/113; C12N 9/22

(52) U.S. Cl. CPC C07K 1/13; C07K 1/22; C12N 2310/20; C12N 15/113; C12N 9/22; C07K 1/36

(57) ABSTRACT

Processes for producing recombinant proteins having low levels of residual host cell proteins. The processes comprise utilizing engineered host cell lines in which specific host cell proteins are tagged with purification tags, wherein the purification tags can be used to remove those specific host cell proteins from the recombinant proteins.

### FIELD

The present disclosure relates to processes for producing recombinant proteins with reduced levels of host cell protein contamination. In particular, the processes comprise expressing recombinant proteins in host cell lines engineered to have tagged host cell proteins, wherein the tagged host cell proteins are tagged with purification tags, such that the purification tags can be used to remove the tagged host cell proteins from the recombinant proteins during purification of the recombinant proteins.

### BACKGROUND

During recombinant protein production, host cells coproduce endogenous proteins related to normal cell functions such as cell growth, proliferation, survival, gene transcription, protein synthesis, and the like. Endogenous host cell proteins also can be released into the cell culture medium as a result of cell death/apoptosis/lysis. All the endogenous proteins co-expressed during recombinant protein production are called host cell proteins (HCPs). HCPs constitute a major part of process-related impurities present in recombinant therapeutic proteins, such as monoclonal antibodies. These HCP impurities can significantly affect efficacy and stability of therapeutic proteins, as well as cause immunogenicity. Moreover, HCPs that copurify with the therapeutic protein can be difficult to remove, resulting in significant downstream processing and increased production costs. For example, it has been estimated that about 80% of monoclonal antibody production costs are due to downstream purification processes. Moreover, to meet regulatory requirements, manufacturers must demonstrate clearance of host cell proteins in the final product to levels in the range of 1 to 100 ppm.

Thus, there is a need for means to reduce or eliminate specific HCPs during therapeutic protein production. For example, therapeutic proteins could be produced in host cell lines comprising HCPs that are tagged with purification tags, wherein the purification tags do not affect the function of the HCPs and the purification tags can be used as tools to remove the tagged HCPs from the therapeutic proteins during downstream purification processes.

### SUMMARY

Among the various aspects of the present disclosure is the provision of processes for producing a recombinant protein product having reduced levels of host cell protein contamination. The processes comprise (a) expressing a recombinant protein in a mammalian cell line engineered to have at least one tagged host cell protein, wherein the at least one tagged host cell protein is tagged with at least one purification tag, and wherein the at least one tagged host cell protein is functionally equivalent to an untagged parental host cell protein; and (b) using the at least one purification tag of the at least one tagged host cell protein to remove the at least one tagged host cell protein from the recombinant protein during purification of the recombinant protein to produce the recombinant protein product, wherein the recombinant protein product has a level of residual host cell protein that is lower than that in a protein product produced by a non-engineered parental mammalian cell line.

Another aspect of the present disclosure encompasses kits for the production of recombinant proteins product having a reduced level of host cell protein contamination. The kits comprise (a) a mammalian cell line engineered to have at least one tagged host cell protein, wherein the at least one tagged host cell protein is tagged with at least one purification tag, and the mammalian cell line is used for expression of a recombinant protein; and (b) means for purifying and/or removing the at least one tagged host cell protein from the recombinant protein during purification of the recombinant protein to produce the recombinant protein product.

Other aspects and iterations of the disclosure are described in more detail below.

### BRIEF DESCRIPTION OF THE DRAWINGS

FIG. 1 presents a schematic showing two different tagging strategies.

FIG. 2A summarizes the 6TG toxicity assay and the HAT assay.

FIG. 2B presents productivity and growth profiles of wild-type, HPRT knockout, and two HPRT tagged cell clones in the presence or absence of 6TG.

FIG. 2C shows productivity and growth profiles of wild-type, HPRT knockout, and two HPRT tagged cell clones in the presence or absence HAT.

### DETAILED DESCRIPTION

The present disclosure provides processes for producing recombinant proteins having reduced levels of host cell protein contamination and engineered host cell lines that can be used to produce the recombinant proteins with low levels of contaminating residual host cell proteins. The host cell lines are engineered such that specific host cell proteins are tagged with “purification” tags. When the engineered host cell lines disclosed herein are used to produce recombinant proteins, the tagged host cell proteins can be removed from the recombinant protein by virtue of their purification tags. As a consequence, the final recombinant protein product has very low levels of residual host cell proteins. Methods for producing said engineered cell lines are provided, as well as kits comprising the engineered cell lines and purification means for removing the tagged host cell proteins from the recombinant proteins.

(I) Process for Producing Recombinant Protein with Reduced Host Cell Protein Levels

One aspect of the present disclosure encompasses processes producing a recombinant protein product having reduced levels of host cell protein contamination. The processes disclosed herein comprise (a) expressing a recombinant protein in a host cell line engineered to have at least one tagged host cell protein, wherein the at least one tagged host cell protein is tagged with at least one purification tag, and wherein the at least one tagged host cell protein is functionally equivalent to an untagged parental host cell protein; and (b) using the at least one purification tag of the at least one tagged host cell protein to remove the at least one tagged host cell protein from the recombinant protein during purification of the recombinant protein to produce the recombinant protein product, wherein the recombinant protein product has a level of residual host cell protein that is lower than that in a protein product produced by a non-engineered parental mammalian cell line.

(a) Host Cell Lines

The processes described herein comprise expressing the recombinant protein in a host cell line engineered to have at least one tagged host cell protein (HCP), wherein the at least one tagged HCP is tagged with at least one purification tag. In general, the tagged HCPs are proteins that are highly abundant, difficult to remove during downstream purification processes, and/or affect product quality (e.g., residual proteases could degrade the biotherapeutic product thereby reducing its efficacy). HCPs with these characteristics are termed “problematic” HCPs. In general, the tagged HCPs are proteins that are essential for cell survival and/or cell function (and, thus, are not good candidates for gene knock-out strategies).

While virtually any host cell protein could be tagged with one or more purification tags, several HCPs are of particular interest. Table A lists some HCPs that could be tagged.

TABLE A
Target HCPs

| Protein | Accession number |
|---|---|
| Clusterin | NW_003614124.1 |
| Matrix metalloproteinase-9 | NW_003613798.1 |
| Matrix metalloproteinase-14 | NW_003614213.1 |
| Matrix metalloproteinase-19 | NW_003614217.1 |
| Hypoxanthine | NW_003613932.1 |
| phosphoribosyltransferase | |
| Secreted protein acidic and rich | NW_003613733.1 |
| in cysteine (SPARC) | |
| Thioredoxin | NW_003614309.1 |
| Thioredoxin Reductase | NW_003614195.1 |

The tagged HCPs comprise at least one purification tag. Non-limiting examples of suitable tags include fluorescent proteins, His tag, poly(His) tag, FLAG (or DDK) tag, Halo tag, AcV5 tag, AU1 tag, AU5 tag, biotin carboxyl carrier protein (BCCP), calmodulin binding protein (CBP), chitin binding domain (CBD), E tag, E2 tag, ECS tag, eXact tag, Glu-Glu tag, glutathione-S-transferase (GST), HA tag, HSV tag, KT3 tag, maltose binding protein (MBP), MAP tag, Myc tag, NE tag, NusA tag, PDZ tag, S tag, S1 tag, SBP tag, Softag 1 tag, Softag 3 tag, Spot tag, Strep tag, SUMO tag, T7 tag, tandem affinity purification (TAP) tag, V5 tag, VSV-G tag, or Xa tag. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein.

The one or more tags can be located at the amino terminal end and/or the carboxy terminal end of the HCP. The function of the HCP is not affected by the tag. Stated another way, the tagged HCP is functionally equivalent to the untagged parental protein.

In some embodiments, the engineered cell line comprises one tagged HCP. In other embodiments, the engineered cell line comprises two tagged HCPs. In further embodiments, the engineered cell line comprises three tagged HCPs. In still other embodiments, the engineered cell line comprises four tagged HCPs. In additional embodiments, the engineered cell line comprises five or more tagged HCPs.

The host cell lines comprising one or more tagged HCPs are genetically engineered to comprise at least one sequence encoding the purification tag that is integrated into a chromosomal sequence encoding the HCP of interest. The tag sequence can be integrated into the chromosomal sequence of interest using targeted endonuclease-mediated genomic editing techniques, which are detailed below in section (III). Targeting endonucleases can be designed to introduce a double-stranded break at a target site in a chromosomal sequence of interest, wherein the double-stranded break can be repaired by a homology-directed repair (HDR) process or a non-homologous end-joining (NHEJ) process. Thus, upon co-introduction of a donor polynucleotide comprising the tag sequence that is flanked by sequence having substantial sequence identity with chromosomal sequence at or near the target site, repair of the double-stranded break leads to integration of the tag sequence into the target site of chromosomal sequence. Generally, the tag sequence is integrated in-frame with the chromosomal sequence encoding the HCP of interest. In-frame means that the open reading frame (ORF) of the chromosomal sequence encoding the HCP is maintained after the insertion of the tag sequence, such that the cell line produces a tagged full-length HCP whose function is similar to that of the untagged HCP.

Thus, the function of the one or more tagged HCPs is maintained in the engineered cell lines. Moreover, the general cell health, cell viability, viable cell density, titer, growth rate, proliferation responses, cell morphology, and/or apoptosis and autophagy levels are similar in the engineered cell lines and their non-engineered parental cells.

In general, the engineered host cell lines are mammalian cell lines. In some embodiments, the engineered cell lines can be derived from human cell lines. Non-limiting examples of suitable human cell lines includes human embryonic kidney cells (HEK293, HEK293T); human connective tissue cells (HT-1080); human cervical carcinoma cells (HELA); human embryonic retinal cells (PER.C6); human kidney cells (HKB-11); human liver cells (Huh-7); human lung cells (W138); human liver cells (Hep G2); human U2-OS osteosarcoma cells, human A549 lung cells, human A-431 epidermal cells, or human K562 bone marrow cells. In other embodiments, the engineered cell lines can be derived from non-human cell lines. Suitable cell lines include, without limit, Chinese hamster ovary (CHO) cells; baby hamster kidney (BHK) cells; mouse myeloma NS0 cells; mouse myeloma Sp2/0 cell; mouse mammary gland C127 cells; mouse embryonic fibroblast 3T3 cells (NIH3T3); mouse B lymphoma A20 cells; mouse melanoma B16 cells; mouse myoblast C2C12 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; or African green monkey kidney (VERO, VERO-76) cells. An extensive list of mammalian cell lines may be found in the American Type Culture Collection catalog (ATCC, Manassas, Va.). In some embodiments, the cell lines disclosed herein are other than mouse cell lines. In certain embodiments, the engineered cell lines are CHO cell lines. Suitable CHO cell lines include, but are not limited to, CHO-K1, CHO-K1SV, CHO GS-/-, CHO S, DG44, DuxxB11, and derivatives thereof.

In various embodiments, the engineered cell lines also can be deficient in glutamine synthase (GS), dihydrofolate reductase (DHFR), hypoxanthine-guanine phosphoribosyltransferase (HPRT), or a combination thereof. For example, the chromosomal sequences encoding GS, DHFR, and/or HPRT can be inactivated. In specific embodiments, all chromosomal sequences encoding GS, DHFR, and/or HPRT are inactivated in the engineered cell lines.

(b) Recombinant Proteins

The processes disclosed herein can be used to produce any recombinant protein. In general, the recombinant protein is heterologous, meaning that the protein is not native to the host cell line. The recombinant protein may be, without limit, a therapeutic protein chosen from an antibody, a fragment of an antibody, a monoclonal antibody, a humanized antibody, a humanized monoclonal antibody, a chimeric antibody, an IgG molecule, an IgG heavy chain, an IgG light chain, an IgA molecule, an IgD molecule, an IgE molecule, an IgM molecule, a vaccine, a growth factor, a cytokine, an interferon, an interleukin, a hormone, a clotting (or coagulation) factor, a blood component, an enzyme, a therapeutic protein, a nutraceutical protein, a functional fragment or functional variant of any of the forgoing, or a fusion protein comprising any of the foregoing proteins and/or functional fragments or variants thereof.

In general, nucleic acid encoding the recombinant protein is introduced into the host cell lines. The nucleic acid can be DNA or RNA, double-stranded or single-stranded, and/or linear or circular. In some embodiments, the nucleic acid encoding the recombinant protein can be located extrachromosomally. That is, the nucleic acid encoding the recombinant protein can be transiently expressed from a plasmid, a cosmid, an artificial chromosome, a minichromosome, or another extrachromosomal construct. In other embodiments, the nucleic acid encoding the recombinant protein can be chromosomally integrated into the genome of the cell. The integration can be random or targeted. Accordingly, the recombinant protein can be stably expressed. In some iterations of this embodiment, the nucleic acid sequence encoding the recombinant protein can be operably linked to an appropriate heterologous expression control sequence (i.e., promoter). In other iterations, the nucleic acid sequence encoding the recombinant protein can be placed under control of an endogenous expression control sequence. The nucleic acid sequence encoding the recombinant protein can be integrated into the genome of the cell line using homologous recombination, targeting endonuclease-mediated genome editing, viral vectors, transposons, plasmids, and other well-known means.

In some embodiments, the nucleic acid encoding the recombinant protein can be linked to sequence encoding hypoxanthine-guanine phosphoribosyltransferase (HPRT), dihydrofolate reductase (DHFR), and/or glutamine synthase (GS), such that HPRT, DHFR, and/or GS may be used as an amplifiable selectable marker. The nucleic acid encoding the recombinant protein also can be linked to sequence encoding at least one antibiotic resistance gene and/or sequence encoding marker proteins such as fluorescent proteins. In some embodiments, the nucleic acid encoding the recombinant protein can be part of an expression construct. The expression constructs or vectors can comprise additional expression control sequences (e.g., enhancer sequences, Kozak sequences, polyadenylation sequences, transcriptional termination sequences, etc.), selectable marker sequences, origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

(c) Expression and Purification of the Recombinant Protein

Means for producing or manufacturing recombinant proteins are well known in the field (see, e.g., “Biopharmaceutical Production Technology”, Subramanian (ed.), 2012, Wiley-VCH; ISBN: 978-3-527-33029-4). Means for purifying the recombinant proteins are well known in the art, and can include clarification (e.g., filtration or centrifugation), affinity chromatography, immunoaffinity chromatography, protein A (or G) chromatography, ion exchange (i.e., cation and/or anion) chromatography, size exclusion chromatography, adsorption chromatography, hydrophobic interaction chromatography, reverse phase chromatography, ultracentrifugation, precipitation, immunoprecipitation, extraction, phase separation, and the like.

The one or more purification tags of the tagged HCPs can be utilized to remove the tagged HCPs from the recombinant protein during downstream production processes or purification processes. For example, HCPs tagged with poly(His) tags can be removed using immobilized metal ion (e.g., nickel or cobalt) affinity chromatography or immunoaffinity (i.e., using antibodies against the poly(His) tag) chromatography. Other tagged HCPs can be removed using suitable affinity chromatography means, immunoaffinity chromatography means, and/or immunoprecipitation means. The tagged HCPs can be removed from the recombinant protein before, during, and/or after purification of the recombinant protein.

Recombinant proteins produced by the processes disclosed herein have reduced levels of HCPs as compared to recombinant proteins produced by non-engineered parental cell lines. In general, the residual levels of HCPs in recombinant proteins produced by the processes disclosed herein are less than 100 ppm, less than 30 ppm, less than 10 ppm, less than 3 ppm, less than 1 ppm, less than 0.3 ppm, less than 0.1 ppm, less than 0.03 ppm, less than 0.01 ppm, less than 0.003, or less than 0.001 ppm, as measured using validated methods in accordance with International Conference on Harmonization (ICG) guidelines. Suitable methods include Western immunoblotting assays, ELISA enzyme assays, one- or two-dimensional SDS polyacrylamide gel electrophoresis (SDS-PAGE), 2D-differential in-gel electrophoresis (DIGE), capillary zone electrophoresis-electrospray ionization-tandem mass spectrometry (CZE-ESI-MS/MS), liquid chromatography-tandem mass spectrometry (LC-MS/MS), two-dimensional-liquid chromatography-tandem mass spectrometry (2D-LC-MS/MS), and the like.

### (II) Kits

A further aspect of the present disclosure provides kits for the production of recombinant proteins, wherein a kit comprises any of the engineered host cell lines detailed above in section (Ia) and means for purifying and/or removing the tagged host cell proteins from the recombinant protein product. Suitable means for purifying the tagged proteins include affinity or immunoaffinity beads, resins, columns, etc., and reagents for affinity chromatography or immunoaffinity chromatography, as well as reagents for immunoprecipitation. The kits can further comprise cell growth media, transfection reagents, selection media, recombinant protein purification means, buffers, and the like. The kits provided herein generally include instructions for growing the cell lines and using them to produce recombinant proteins. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### (III) Methods for Preparing Engineered Host Cell Lines

Yet another aspect of the present disclosure provides methods for preparing or engineering the host cell lines comprising one or more tagged HCPs, which are described above in section (Ia). In general, the engineered cell lines are prepared using a targeting endonuclease-mediated genome modification process. Such a process comprises introducing into a parental cell line of interest at least one targeting endonuclease engineered to cleave a target site in a chromosomal sequence encoding the HCP of interest or nucleic acid encoding said targeting endonuclease and at least one donor polynucleotide. A donor polynucleotide comprises a tag sequence to be integrated into the chromosomal sequence encoding the HCP of interest, wherein the tag sequence in the donor polynucleotide is flanked by sequences having substantial sequence identity with sequence at or near the target site in the chromosomal sequence. The parental cell line is then maintained under conditions such that a double-stranded break introduced at the target site by the targeting endonuclease is repaired by a homology-directed process such that the tag sequence in the donor polynucleotide is integrated in-frame at the 5′ end and/or 3′ end of the chromosomal sequence encoding the HCP of interest.

(a) Targeting Endonucleases

A variety of targeting endonucleases can be used to integrate tag sequences into chromosomal sequences encoding the HCPs of interest. The targeting endonuclease can be a naturally-occurring protein or an engineered protein. Suitable targeting endonucleases include, without limit, zinc finger nucleases (ZFNs), CRISPR nucleases, transcription activator-like effector (TALE) nucleases (TALENs), meganucleases, chimeric nucleases, site-specific endonucleases, and artificial targeted DNA double strand break inducing agents.

(i) Zinc Finger Nucleases

In specific embodiments, the targeting endonuclease can be a pair of zinc finger nucleases (ZFNs). ZFNs bind to specific targeted sequences and introduce a double-stranded break into a targeted cleavage site. Typically, a ZFN comprises a DNA binding domain (i.e., zinc fingers) and a cleavage domain (i.e., nuclease), each of which is described below.

DNA binding domain. A DNA binding domains or the zinc fingers can be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 can be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences as well as designing zinc finger binding domains are known in the art. For example, tools for identifying potential target sites in DNA sequences can be found at zincfingertools.org. Tools for designing zinc finger binding domains can be found at zifit.partners.org/ZiFiT. (See also, Mandell et al. (2006) Nuc. Acid Res. 34:W516-W523; Sander et al. (2007) Nuc. Acid Res. 35:W599-W605.)

A zinc finger binding domain can be designed to recognize and bind a DNA sequence ranging from about 3 nucleotides to about 21 nucleotides in length. In one embodiment, the zinc finger binding domain can be designed to recognize and bind a DNA sequence ranging from about 9 to about 18 nucleotides in length. In general, the zinc finger binding domains of the zinc finger nucleases used herein comprise at least three zinc finger recognition regions or zinc fingers, wherein each zinc finger binds 3 nucleotides. In one embodiment, the zinc finger binding domain comprises four zinc finger recognition regions. In another embodiment, the zinc finger binding domain comprises five zinc finger recognition regions. In still another embodiment, the zinc finger binding domain comprises six zinc finger recognition regions. A zinc finger binding domain can be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region include phage display and two-hybrid systems, which are described in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227, the entire disclosure of which is incorporated herein by reference.

Zinc finger binding domains and methods for design and construction of fusion proteins (and polynucleotides encoding same) are known to those of skill in the art and are described in detail in, for example, U.S. Pat. No. 7,888,121, which is incorporated by reference herein in its entirety. Zinc finger recognition regions and/or multi-fingered zinc finger proteins can be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

Cleavage domain. A zinc finger nuclease also includes a cleavage domain. The cleavage domain portion of the zinc finger nuclease can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) can be used as a source of cleavage domains.

A cleavage domain also can be derived from an enzyme or portion thereof, as described above, that requires dimerization for cleavage activity. Two zinc finger nucleases can be required for cleavage, as each nuclease comprises a monomer of the active enzyme dimer. Alternatively, a single zinc finger nuclease can comprise both monomers to create an active enzyme dimer. As used herein, an “active enzyme dimer” is an enzyme dimer capable of cleaving a nucleic acid molecule. The two cleavage monomers can be derived from the same endonuclease (or functional fragments thereof), or each monomer can be derived from a different endonuclease (or functional fragments thereof).

When two cleavage monomers are used to form an active enzyme dimer, the recognition sites for the two zinc fingers are preferably disposed such that binding of the two zinc fingers to their respective recognition sites places the cleavage monomers in a spatial orientation to each other that allows the cleavage monomers to form an active enzyme dimer, e.g., by dimerizing. As a result, the near edges of the recognition sites can be separated by about 5 to about 18 nucleotides. For instance, the near edges can be separated by about 5, 6, 7, 8, 9, 10, 11, 12, 13, 14, 15, 16, 17 or 18 nucleotides. It will however be understood that any integral number of nucleotides or nucleotide pairs can intervene between two recognition sites (e.g., from about 2 to about 50 nucleotide pairs or more). The near edges of the recognition sites of the zinc finger nucleases, such as for example those described in detail herein, can be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31978-31982. Thus, a zinc finger nuclease can comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is FokI. This particular enzyme is active as a dimer (Bitinaite et al. (1998) Proc. Natl. Acad. Sci. USA 95: 10, 570-10, 575). Accordingly, for the purposes of the present disclosure, the portion of the FokI enzyme used in a zinc finger nuclease is considered a cleavage monomer. Thus, for targeted double-stranded cleavage using a FokI cleavage domain, two zinc finger nucleases, each comprising a FokI cleavage monomer, can be used to reconstitute an active enzyme dimer. Alternatively, a single polypeptide molecule containing a zinc finger binding domain and two FokI cleavage monomers can also be used.

In certain embodiments, the cleavage domain comprises one or more engineered cleavage monomers that minimize or prevent homodimerization. By way of non-limiting example, amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains. Exemplary engineered cleavage monomers of FokI that form obligate heterodimers include a pair in which a first cleavage monomer includes mutations at amino acid residue positions 490 and 538 of FokI and a second cleavage monomer that includes mutations at amino-acid residue positions 486 and 499.

Thus, in one embodiment of the engineered cleavage monomers, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers can be prepared by mutating positions 490 from E to K and 538 from I to K in one cleavage monomer to produce an engineered cleavage monomer designated “E490K:1538K” and by mutating positions 486 from Q to E and 499 from I to K in another cleavage monomer to produce an engineered cleavage monomer designated “Q486E:I499K.” The above described engineered cleavage monomers are obligate heterodimer mutants in which aberrant cleavage is minimized or abolished. Engineered cleavage monomers can be prepared using a suitable method, for example, by site-directed mutagenesis of wild-type cleavage monomers (FokI) as described in U.S. Pat. No. 7,888,121, which is incorporated herein in its entirety.

Additional domains. In some embodiments, the zinc finger nuclease can further comprise at least one nuclear localization sequence (NLS). A NLS is an amino acid sequence which facilitates targeting the zinc finger nuclease protein into the nucleus to introduce a double stranded break at the target sequence in the chromosome. Nuclear localization signals are known in the art (see, e.g., Lange et al., J. Biol. Chem., 2007, 282:5101-5105). Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO:12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO:16), NQSSNFGPMKGGNFGGRSSGPYGGGGQYFAKPRNQGGY (SEQ ID NO:17), and RMRIZFKNKGKDTAELRRRRVEVSVELRKAKKDEQILKRRNV (SEQ ID NO:18). The NLS can be located at the N-terminus, the C-terminus, or in an internal location of the zinc finger nuclease.

In additional embodiments, the zinc finger nuclease can also comprise at least one cell-penetrating domain. Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO:24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO:28), KALAWEAKLAKALAKALAKHLAKALAKALKCEA (SEQ ID NO:29), and RQIKIWFQNRRMKWKK (SEQ ID NO:30). The cell-penetrating domain can be located at the N-terminus, the C-terminus, or in an internal location of the zinc finger nuclease.

In still other embodiments, the zinc finger nuclease can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, poly(His) tag, FLAG (or DDK) tag, Halo tag, AcV5 tag, AU1 tag, AU5 tag, biotin carboxyl carrier protein (BCCP), calmodulin binding protein (CBP), chitin binding domain (CBD), E tag, E2 tag, ECS tag, eXact tag, Glu-Glu tag, glutathione-S-transferase (GST), HA tag, HSV tag, KT3 tag, maltose binding protein (MBP), MAP tag, Myc tag, NE tag, NusA tag, PDZ tag, S tag, S1 tag, SBP tag, Softag 1 tag, Softag 3 tag, Spot tag, Strep tag, SUMO tag, T7 tag, tandem affinity purification (TAP) tag, thioredoxin (TRX), V5 tag, VSV-G tag, and Xa tag. tandem affinity purification (TAP) tag, thioredoxin (TRX), V5 tag, VSV-G tag, and Xa tag. The marker domain can be located at the N-terminus, the C-terminus, or in an internal location of the zinc finger nuclease.

The at least one nuclear localization signal, at least one cell-penetrating domain, and/or at least one marker domain can be linked directly to the zinc finger nuclease via one or more chemical bonds (e.g., covalent bonds). Alternatively, the at least one nuclear localization signal, at least one cell-penetrating domain, and/or at least one marker domain, can be linked indirectly to the zinc finger nuclease via one or more linkers. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In some embodiments, the linker can be a peptide linker. The peptide linker can be a flexible amino acid linker or a rigid amino acid linker. Additional examples of suitable linkers are well known in the art and programs to design linkers are readily available (Crasto et al., Protein Eng., 2000, 13(5):309-312).

CRISPR Ribonucleoproteins (RNPs)

In other embodiments, the targeting endonuclease can be a Clustered Regularly Interspersed Short Palindromic Repeat (CRISPR) nuclease. CRISPR nucleases are RNA-guided nucleases derived from bacterial or archaeal CRISPR/CRISPR-associated (Cas) systems. A CRISPR RNP system comprises a CRISPR nuclease and a guide RNA.

Nuclease. The CRISPR nuclease can be derived from a type I (i.e., IA, IB, IC, ID, IE, or IF), type II (i.e., IIA, IIB, or IIC), type III (i.e., IIIA or IIIB), type V, or type VI CRISPR system, which are present in various bacteria and archaea. For example, the CRISPR nuclease can be from Streptococcus sp. (e.g., S. pyogenes, S. thermophilus, S. pasteurianus), Campylobacter sp. (e.g., Campylobacterjejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lachnospiraceae sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., Thermosipho sp., or Verrucomicrobia sp. In other embodiments, the CRISPR nuclease can be derived from an archaeal CRISPR system, a CRISPR/CasX system, or a CRISPR/CasY system (Burstein et al., Nature, 2017, 542(7640):237-241).

In some embodiments, the CRISPR nuclease can be derived from a type II CRISPR nuclease. For example, the type II CRISPR nuclease can be a Cas9 protein. Suitable Cas9 nucleases include Streptococcus pyogenes Cas9 (SpCas9), Francisella novicida Cas9 (FnCas9), Staphylococcus aureus (SaCas9), Streptococcus thermophilus Cas9 (StCas9), Streptococcus pasteurianus (SpaCas9), Campylobacter jejuni Cas9 (CjCas9), Neisseria meningitis Cas9 (NmCas9), or Neisseria cinerea Cas9 (NcCas9). In other embodiments, the CRISPR nuclease can be derived from a type V CRISPR nuclease, such as a Cpf1 nuclease. Suitable Cpf1 nucleases include Francisella novicida Cpf1 (FnCpf1), Acidaminococcus sp. Cpf1 (AsCpf1), or Lachnospiraceae bacterium ND2006 Cpf1 (LbCpf1). In yet another embodiment, the CRISPR nuclease can be derived from a type VI CRISPR nuclease, e.g., Leptotrichia wadei Cas13a (LwaCas13a) or Leptotrichia shahii Cas13a (LshCas13a).

The CRISPR nuclease can be a wild type CRISPR nuclease, a modified CRISPR nuclease, or a fragment of a wild type or modified CRISPR nuclease. The CRISPR nuclease can be modified to increase nucleic acid binding affinity and/or specificity, alter enzymatic activity, and/or change another property of the protein. For example, nuclease (i.e., DNase, RNase) domains of the CRISPR nuclease can be modified, deleted, or inactivated. The CRISPR nuclease can be truncated to remove domains that are not essential for the function of the nuclease.

CRISPR nucleases comprise two nuclease domains. For example, a Cas9 nuclease comprises a HNH domain, which cleaves the guide RNA complementary strand, and a RuvC domain, which cleaves the non-complementary strand; a Cpf1 nuclease comprises a RuvC domain and a NUC domain; and a Cas13a nuclease comprises two HNEPN domains. When both nuclease domains are functional, CRISPR nuclease introduces a double-stranded break. Either nuclease domain can be inactivated by one or more mutations and/or deletions, thereby creating a variant that introduces a single-strand break in one strand of the double-stranded sequence. For example, one or more mutations in the RuvC domain of Cas9 nuclease (e.g., D10A, D8A, E762A, and/or D986A) results in an HNH nickase that nicks the guide RNA complementary strand; and one or more mutations in the HNH domain of Cas9 nuclease (e.g., H840A, H559A, N854A, N856A, and/or N863A) results in a RuvC nickase that nicks the guide RNA non-complementary strand. Comparable mutations can convert Cpf1 and Cas13a nucleases to nickases. Two CRISPR nickases targeted to opposites strands of a chromosomal sequence (via a pair of offset guide RNAs) can be used in combination to create a double-stranded break in the chromosomal sequence. Dual CRISPR nickase RNPs can increase target specificity and reduce off target effects.

Additional domains. The CRISPR nuclease can further comprise at least one nuclear localization sequence (NLS). A NLS is an amino acid sequence which facilitates targeting the zinc finger nuclease protein into the nucleus to introduce a double stranded break at the target sequence in the chromosome. Nuclear localization signals are known in the art (see, e.g., Lange et al., J. Biol. Chem., 2007, 282:5101-5105). Non-limiting examples of nuclear localization signals include PKKKRKV (SEQ ID NO:1), PKKKRRV (SEQ ID NO:2), KRPAATKKAGQAKKKK (SEQ ID NO:3), YGRKKRRQRRR (SEQ ID NO:4), RKKRRQRRR (SEQ ID NO:5), PAAKRVKLD (SEQ ID NO:6), RQRRNELKRSP (SEQ ID NO:7), VSRKRPRP (SEQ ID NO:8), PPKKARED (SEQ ID NO:9), PQPKKKPL (SEQ ID NO:10), SALIKKKKKMAP (SEQ ID NO:11), PKQKKRK (SEQ ID NO:12), RKLKKKIKKL (SEQ ID NO:13), REKKKFLKRR (SEQ ID NO:14), KRKGDEVDGVDEVAKKKSKK (SEQ ID NO:15), RKCLQAGMNLEARKTKK (SEQ ID NO:16), NQSSNFGPMKGGNFGGRSSGPYGGGGQYFAKPRNQGGY (SEQ ID NO:17), and RMRIZFKNKGKDTAELRRRRVEVSVELRKAKKDEQILKRRNV (SEQ ID NO:18). The NLS can be located at the N-terminus, the C-terminus, or in an internal location of the CRISPR nuclease.

In additional embodiments, the CRISPR nuclease can also comprise at least one cell-penetrating domain. Examples of suitable cell-penetrating domains include, without limit, GRKKRRQRRRPPQPKKKRKV (SEQ ID NO:19), PLSSIFSRIGDPPKKKRKV (SEQ ID NO:20), GALFLGWLGAAGSTMGAPKKKRKV (SEQ ID NO:21), GALFLGFLGAAGSTMGAWSQPKKKRKV (SEQ ID NO:22), KETWWETWWTEWSQPKKKRKV (SEQ ID NO:23), YARAAARQARA (SEQ ID NO:24), THRLPRRRRRR (SEQ ID NO:25), GGRRARRRRRR (SEQ ID NO:26), RRQRRTSKLMKR (SEQ ID NO:27), GWTLNSAGYLLGKINLKALAALAKKIL (SEQ ID NO:28), KALAWEAKLAKALAKALAKHLAKALAKALKCEA (SEQ ID NO:29), and RQIKIWFQNRRMKWKK (SEQ ID NO:30). The cell-penetrating domain can be located at the N-terminus, the C-terminus, or in an internal location of the CRISPR protein.

In still other embodiments, the CRISPR nuclease can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, poly(His) tag, FLAG (or DDK) tag, Halo tag, AcV5 tag, AU1 tag, AU5 tag, biotin carboxyl carrier protein (BCCP), calmodulin binding protein (CBP), chitin binding domain (CBD), E tag, E2 tag, ECS tag, eXact tag, Glu-Glu tag, glutathione-S-transferase (GST), HA tag, HSV tag, KT3 tag, maltose binding protein (MBP), MAP tag, Myc tag, NE tag, NusA tag, PDZ tag, S tag, S1 tag, SBP tag, Softag 1 tag, Softag 3 tag, Spot tag, Strep tag, SUMO tag, T7 tag, tandem affinity purification (TAP) tag, thioredoxin (TRX), V5 tag, VSV-G tag, and Xa tag. The marker domain can be located at the N-terminus, the C-terminus, or in an internal location of the CRISPR nuclease.

The at least one nuclear localization signal, at least one cell-penetrating domain, and/or at least one marker domain can be linked directly to the CRISPR nuclease via one or more chemical bonds (e.g., covalent bonds). Alternatively, the at least one nuclear localization signal, at least one cell-penetrating domain, and/or at least one marker domain, can be linked indirectly to the CRISPR nuclease via one or more linkers. Suitable linkers include amino acids, peptides, nucleotides, nucleic acids, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. In some embodiments, the linker can be a peptide linker. The peptide linker can be a flexible amino acid linker or a rigid amino acid linker. Additional examples of suitable linkers are well known in the art and programs to design linkers are readily in the art.

Guide RNA. A CRISPR nuclease is guided to its target site by a guide RNA. The guide RNA hybridizes with the target site and interacts with the CRISPR nuclease to direct the CRISPR nuclease to the target site in the chromosomal sequence. The target site has no sequence limitation except that the sequence is bordered by a protospacer adjacent motif (PAM). CRISPR proteins from different bacterial species recognize different PAM sequences. For example, PAM sequences include 5′-NGG (SpCas9, FnCAs9), 5′-NGRRT (SaCas9), 5′-NNAGAAW (StCas9), 5′-NNNNGATT (NmCas9), 5-NNNNRYAC (CjCas9), and 5′-TTTV (Cpf1), wherein N is defined as any nucleotide, R is defined as either G or A, W is defined as either A or T, Y is defined an either C or T, and V is defined as A, C, or G. Cas9 PAMs are located 3′ of the target site, and cpf1 PAMs are located 5′ of the target site.

A guide RNA comprises three regions: a first region at the 5′ end that is complementary to sequence at the target site, a second internal region that forms a stem loop structure, and a third 3′ region that remains essentially single-stranded. The first region of each guide RNA is different such that each guide RNA guides a CRISPR nuclease to a specific target site. The second and third regions (also called the scaffold region) of each guide RNA can be the same in all guide RNAs.

The first region of the guide RNA is complementary to sequence (i.e., protospacer sequence) at the target site such that the first region of the guide RNA can base pair with sequence at the target site. The complementarity between the first region (i.e., crRNA) of the guide RNA and the target sequence can be at least 80%, at least 85%, at least 90%, at least 95%, or more. In general, there are no mismatches between the sequence of the first region of the guide RNA and the sequence at the target site (i.e., the complementarity is total). In various embodiments, the first region of the guide RNA can comprise from about 10 nucleotides to more than about 25 nucleotides. For example, the region of base pairing between the first region of the guide RNA and the target site in the chromosomal sequence can be about 10, 11, 12, 13, 14, 15, 16, 17, 18, 19, 20, 22, 23, 24, 25, or more than 25 nucleotides in length. In exemplary embodiments, the first region of the guide RNA is about 19, 20, or 21 nucleotides in length.

The guide RNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The guide RNA also comprises a third region at the 3′ end that remains essentially single-stranded. Thus, the third region has no complementarity to any chromosomal sequence in the cell of interest and has no complementarity to the rest of the guide RNA. The length of the third region can vary. In general, the third region is more than about 4 nucleotides in length. For example, the length of the third region can range from about 5 to about 60 nucleotides in length.

The combined length of the second and third regions (or scaffold) of the guide RNA can range from about 30 to about 120 nucleotides in length. In one aspect, the combined length of the second and third regions of the guide RNA range from about 70 to about 100 nucleotides in length.

In some embodiments, the guide RNA comprises one molecule comprising all three regions. In other embodiments, the guide RNA can comprise two separate molecules. The first RNA molecule can comprise the first (5′) region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence to form a functional guide RNA.

(iii) Other Targeting Endonucleases

In further embodiments, the targeting endonuclease can be a meganuclease. Meganucleases are endodeoxyribonucleases characterized by long recognition sequences, i.e., the recognition sequence generally ranges from about 12 base pairs to about 40 base pairs. As a consequence of this requirement, the recognition sequence generally occurs only once in any given genome. Among meganucleases, the family of homing endonucleases named LAGLIDADG has become a valuable tool for the study of genomes and genome engineering (see, e.g., Arnould et al., 2011, Protein Eng Des Sel, 24(1-2):27-31). Other suitable meganucleases include I-Crel and I-Dmol. A meganuclease can be targeted to a specific chromosomal sequence by modifying its recognition sequence using techniques well known to those skilled in the art.

In additional embodiments, the targeting endonuclease can be a transcription activator-like effector (TALE) nuclease. TALEs are transcription factors from the plant pathogen Xanthomonas that can be readily engineered to bind new DNA targets. TALEs or truncated versions thereof may be linked to the catalytic domain of endonucleases such as FokI to create targeting endonuclease called TALE nucleases or TALENs (Sanjana et al., 2012, Nat Protoc, 7(1):171-192) and Arnould et al., 2011, Protein Engineering, Design & Selection, 24(1-2):27-31).

In alternate embodiments, the targeting endonuclease can be chimeric nuclease. Non-limiting examples of chimeric nucleases include ZF-meganucleases, TAL-meganucleases, Cas9-FokI fusions, ZF-Cas9 fusions, TAL-Cas9 fusions, and the like. Persons skilled in the art are familiar with means for generating such chimeric nuclease fusions.

In still other embodiments, the targeting endonuclease can be a site-specific endonuclease. In particular, the site-specific endonuclease can be a “rare-cutter” endonuclease whose recognition sequence occurs rarely in a genome. Alternatively, the site-specific endonuclease can be engineered to cleave a site of interest (Friedhoff et al., 2007, Methods Mol Biol 352:1110123). Generally, the recognition sequence of the site-specific endonuclease occurs only once in a genome. In alternate further embodiments, the targeting endonuclease can be an artificial targeted DNA double strand break inducing agent.

(b) Donor Polynucleotide

The method for preparing the engineered cell lines comprising tagged HCPs further comprises introducing into the parental cell line at least one donor polynucleotide comprising the tag sequence. A donor polynucleotide comprises not only the tag sequence, but also comprises an upstream sequence and a downstream sequence. The upstream and downstream sequences flank the tag sequence in the donor polynucleotide. Furthermore, the upstream and downstream sequences share substantial sequence identity with sequence at either side of the target site in the chromosomal sequence encoding the HCP of interest.

The upstream and downstream sequences in the donor polynucleotide are selected to promote recombination between the target site in the chromosomal sequence and the donor polynucleotide. The upstream sequence, as used herein, refers to a nucleic acid sequence that shares sequence similarity with the chromosomal sequence upstream of the target site. Similarly, the downstream sequence refers to a nucleic acid sequence that shares sequence similarity with the chromosomal sequence downstream of the target site. The upstream and downstream sequences in the donor polynucleotide can have about 75%, 80%, 85%, 90%, 95%, or 100% sequence identity with sequence near the target site in the chromosomal sequence. In other embodiments, the upstream and downstream sequences in the donor polynucleotide can have about 95%, 96%, 97%, 98%, 99%, or 100% sequence identity with sequence near the target site in the chromosomal sequence. In a specific embodiment, the upstream and downstream sequences in the donor polynucleotide have about 99% or 100% sequence identity with sequence near the target site in the chromosomal sequence.

An upstream or downstream sequence can comprise from about 10 nucleotides to about 2500 nucleotides. In one embodiment, an upstream or downstream sequence can comprise about 30, 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 nucleotides. In specific embodiments, the upstream or downstream sequences can comprise from about 200 nucleotides to about 2000 nucleotides, or from about 600 nucleotides to about 1000 nucleotides. In other embodiments, the upstream or downstream sequences can comprise from about 20 nucleotides to about 200 nucleotides.

The length of the donor polynucleotide can and will vary. For example, the polynucleotide can range from about 20 nucleotides in length up to about 200,000 nucleotides in length. In various embodiments, the polynucleotide can range from about 20 nucleotides to about 100 nucleotides in length, from about 100 nucleotides to about 1000 nucleotides in length, from about 1000 nucleotides to about 10,000 nucleotides in length, from about 10,000 nucleotides to about 100,000 nucleotides in length, or from about 100,000 nucleotides to about 200,000 nucleotides in length.

Typically, the donor polynucleotide is DNA. The DNA can be single-stranded or double stranded. The DNA can be linear or circular. In some embodiments, the donor polynucleotide can be an single-stranded, linear oligonucleotide comprising less than about 200 nucleotides. In other embodiments, the donor polynucleotide can be part of a vector. Suitable vectors include DNA plasmids, viral vectors, bacterial artificial chromosomes (BAC), and yeast artificial chromosomes (YAC). In still other embodiments, the donor polynucleotide can be a PCR fragment or a nucleic acid complexed with a delivery vehicle such as a liposome or poloxamer.

(c) Delivery to the Cell

The method comprises introducing the targeting endonuclease(s) and donor polynucleotide(s) into the parental cell line of interest. A targeting endonuclease can be introduced into the cells as a purified isolated protein or as a nucleic acid encoding the targeting endonuclease. The nucleic acid can be DNA or RNA. In embodiments in which the encoding nucleic acid is mRNA, the mRNA may be 5′ capped and/or 3′ polyadenylated. In embodiments in which the encoding nucleic acid is DNA, the DNA can be linear or circular. The nucleic acid can be part of a plasmid or viral vector, wherein the encoding DNA can be operably linked to a suitable promoter. Those skilled in the art are familiar with appropriate vectors, promoters, other control elements, and means of introducing the vector into the cell of interest. In embodiments in which targeting endonuclease is a CRISPR nuclease, the CRISPR nuclease system can be introduced into the cell as a gRNA-protein complex.

The targeting endonuclease molecule(s) and donor polynucleotide(s) can be introduced into the cells by a variety of means. Suitable delivery means include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In a specific embodiment, the targeting endonuclease molecule(s) and donor polynucleotide(s) are introduced into the cell by nucleofection.

The donor polynucleotide(s) can be introduced into the cells at the same time as the targeting endonuclease molecule(s). Alternatively, the donor polynucleotide(s) and the targeting endonuclease molecule(a) can be introduced into the cell sequentially. The ratio of the targeting endonuclease molecule(s) to the donor polynucleotide(s) can and will vary. In general, the ratio of targeting endonuclease molecule(s) to donor polynucleotide(s) ranges from about 1:10 to about 10:1. In various embodiments, the ratio of the targeting endonuclease molecule(s) to donor polynucleotide(s)can may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio is about 1:1.

(d) Culturing the Cell

The method further comprises maintaining the cell under appropriate conditions such that the targeting endonuclease introduces a double-stranded break and the double-stranded break is repaired by a homology-directed repair process such that the tag sequence of the donor polynucleotide is integrated into chromosomal sequence encoding the HCP of interest. In embodiments in which nucleic acid(s) encoding the targeting endonuclease(s) is introduced into the cell, the method comprises maintaining the cell under appropriate conditions such that the cell expresses the targeting endonuclease(s).

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon confirmation that the chromosomal sequence of interest has been tagged, single cell clones can be isolated and genotyped (via DNA sequencing and/or protein analyses). Cells comprising one tagged chromosomal sequence can undergo one or more additional rounds of targeted genome modification to tag additional chromosomal sequences, thereby creating cells lies comprising multiple tagged HCPs.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “endogenous sequence” refers to a chromosomal sequence that is native to the cell.

The term “exogenous sequence” refers to a chromosomal sequence that is not native to the cell, or a chromosomal sequence that is moved to a different chromosomal location.

An “engineered” or “genetically modified” cell refers to a cell in which the genome has been modified or engineered, i.e., the cell contains at least chromosomal sequence that has been engineered to contain an insertion of at least one nucleotide, a deletion of at least one nucleotide, and/or a substitution of at least one nucleotide.

The terms “genome modification” and “genome editing” refer to processes by which a specific chromosomal sequence is changed such that the chromosomal sequence is modified. The chromosomal sequence may be modified to comprise an insertion of at least one nucleotide, a deletion of at least one nucleotide, and/or a substitution of at least one nucleotide. The modified chromosomal sequence is inactivated such that no product is made. Alternatively, the chromosomal sequence can be modified such that an altered product is made.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties. In general, an analog of a particular nucleotide has the same base-pairing specificity; i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site” or “target sequence” refer to a nucleic acid sequence that defines a portion of a chromosomal sequence to be modified or edited and to which a targeting endonuclease is engineered to recognize and bind, provided sufficient conditions for binding exist.

The terms “upstream” and “downstream” refer to locations in a nucleic acid sequence relative to a fixed position. Upstream refers to the region that is 5′ (i.e., near the 5′ end of the strand) to the position and downstream refers to the region that is 3′ (i.e., near the 3′ end of the strand) to the position.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

The following examples illustrate certain aspects of the invention.

### Example 1

### Identification of Contaminating Host Cell Proteins

Mass spectroscopy was used to identify HCPs produced by several CHO parental cell lines. Fed-batch supernatants were collected from different parental cell lines and analyzed by LC-MS/MS. Similarly, sample eluates after a Pro A capture step were analyzed to identify proteins that had associated with the column. In a second approach, HCP profiles were followed from a recombinant expressing clone through downstream purification steps. “Problematic HCPs” were characterized as falling in one of the following three categories (i) highly abundant, (ii) difficult to remove during downstream processing and (iii) impacting product quality. Given the large number of proteins identified in each sample, principal component analysis (PCA) was performed to emphasize variations and mine data patterns. Table 1 lists some identified “problematic” HCPs that were identified and their characteristics.

TABLE 1
Identified Host Cell Proteins

| | | Difficult | Affects |
|---|---|---|---|
| | | to | Product |
| Host Cell Protein | Abundance | Purify | Quality |
| Cathepsin D | + | + | + |
| Carboxypeptidase D | + | | + |
| Lipoprotein lipase | + | + | + |
| Phospholipase B-like 2 | + | + | + |
| Serine protease | + | + | + |
| Nidogen 1 | | + | |
| Clusterin | + | + | |
| Metalloproteinase inhibitor | | | + |
| 1 | | | |
| Thioredoxin & thioredoxin | | | + |
| reductase | | | |
| Peroxidasin | | + | + |
| Hypoxanthine | | | |
| phosphoribosyltransferase | | | |

Several proteases were identified as candidates for gene editing. Proteases originating from the host cells are active in the cell culture medium and can influence the product quality. Their proteolytic activity may degrade the recombinantly expressed polypeptide also referred to as “clipping”, thereby rendering a potentially immunogenic and altered, e.g., non- or less functional therapeutic protein. Identified HCPs were further categorized as essential or non-essential to host cell growth and productivity.

### Example 2

### Tagging Hypoxanthine Phosphoribosyltransferase Gene in CHO Cells

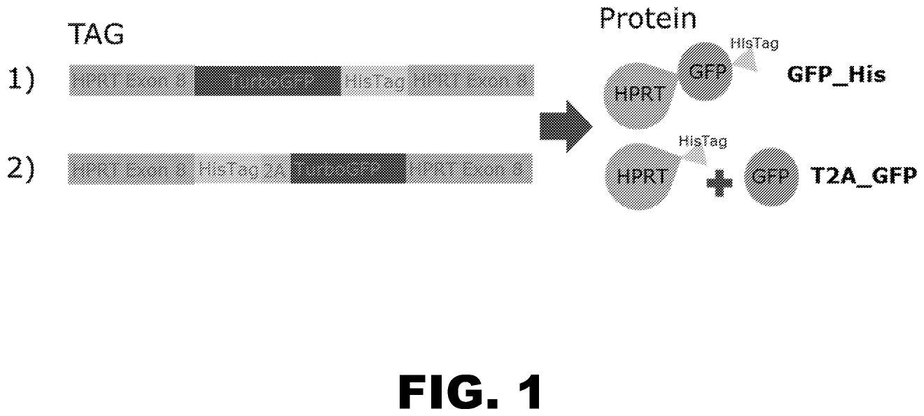

CHO cells were transfected with Cas9 constructs comprising gene-specific gRNA designed to target exon 8 of the hypoxanthine phosphoribosyltransferase (HPRT) and a donor sequence encoding TurboGFP-His tag (GFP_His) or His tag-protein 2A-turboGFP (T2A_GFP). The experiment was designed to insert the tag sequence in the correct reading frame without altering the HPRT sequence. In-fame insertion of the donor sequence immediately upstream of the stop codon of the HPRT coding sequence results in expression (via the endogenous HPRT promoter) of HPRT tagged at the C-terminal end with GFP and the His tag or HPRT tagged at the C-terminal end with the His tag and a separate GFP (see FIG. 1). Clones were isolated and sequenced. The results are presented in Table 1 below.

TABLE 1
Summary of Clones

| | Correct | Modified GFP | Modified HPRT |
|---|---|---|---|
| GFP-His clones | 12 | 11 | 0 |
| T2A_GFP | 27 | 3 | 0 |

One clone (GFP_Histag clone D02) had a large 27 bp deletion in the 5′ region of the GFP sequence. This clone (D02) and a clone having the correct sequence (GFP_Histag clone A04) were tested to determine whether HPRT function was maintained after insertion of the tag.

### Example 3

### Functional Analysis of the Tagged HPRT Clones

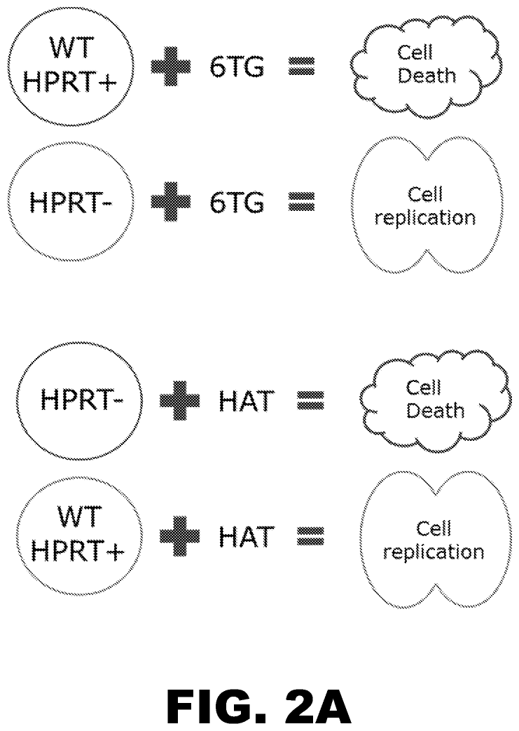

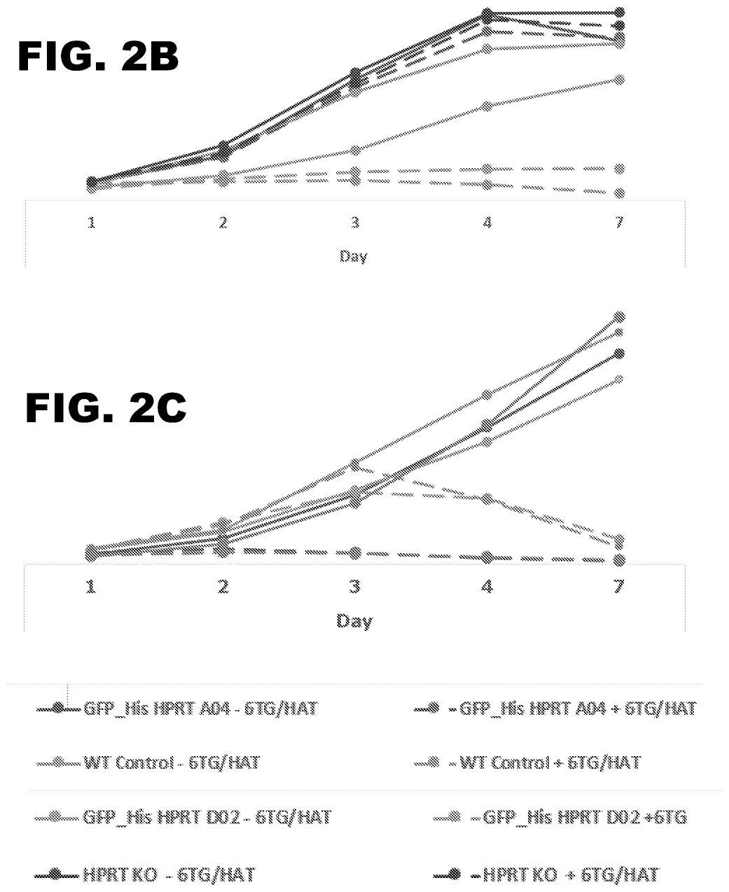

HPRT function was tested via two different assays. 1) 6TG toxicity assay. 6TG is a cytotoxic thio analogue of guanine. In culture, HPRT converts 6TG to 6-thioguanine nucleotides (6-TGNs), which cause cell death once incorporated into DNA during S-phase. Wild-type CHO cells with functional HPRT protein will die in the presence of 6TG, and CHO cells with dysfunctional HPRT protein will survive and divide in presence of 6TG (FIG. 2A). A range of 6TG concentrations were tested and the lowest concentration that killed wild-type cells was used to test HPRT function in the tagged clones. 2) HAT (hypoxanthine-aminopterin-thymidine) assay. Aminopterin is a powerful folate metabolism inhibitor which completely blocks de novo DNA synthesis. In the presence of aminopterin, cells try to synthesize DNA using a salvage pathway. With the addition of hypoxanthine and thymidine DNA intermediates, the cell is able to synthesize DNA in the presence of aminopterin, provided two important enzymes are functional. These two enzymes are thymidine kinase and HPRT. Wild-type CHO cells with functional HPRT protein will survive and replicate in the presence of HAT, but CHO B)cells with dysfunctional HPRT protein will die in the presence of HAT (FIG. 2A).

Wild-type cells, HPRT knockout cells, GFP_Histag clone D02 (GFP deletion), and GFP_Histag clone A04 (correct insertion) were exposed to 6TG or HAT and cell viability and viable cell density were measured each day for 5 days. The results are presented in FIG. 2B (6TG assay) and FIG. 2C (HAT assay). GFP_Histag clone D02 acted more like wild-type cell, suggesting some HPRT function. In contrast, GFP_Histag clone A04 was similar to the HPRT knockout cells.

### Example 4

### Purification of Tagged HPRT Proteins

A 7 day batch assay will be set up using GFP_Histag and T2A_GFP clones of interest. The supernatants will be collected, filtered, and purified using Histag affinity chromatography. The purified protein sample will be assayed for the presence of GFP, Histag, and HPRT.

1. A process for producing a recombinant protein product having a reduced level of host cell protein contamination, the process comprising:
a) expressing a recombinant protein in a mammalian cell line engineered to have at least one tagged host cell protein, wherein the at least one tagged host cell protein is tagged with at least one purification tag, and wherein the at least one tagged host cell protein is functionally equivalent to an untagged parental host cell protein; and
b) using the at least one purification tag of the at least one tagged host cell protein to remove the at least one tagged host cell protein from the recombinant protein during purification of the recombinant protein to produce the recombinant protein product, wherein the recombinant protein product has a level of residual host cell protein that is lower than that in a protein product produced by a non-engineered parental mammalian cell line.

2. The process of claim 1, wherein the at least one tagged protein is an essential protein.

3. The process of claim 2, wherein the essential protein is chosen from clusterin, matrix metalloproteinase-9, matrix metalloproteinase-14, matrix metalloproteinase-19, hypoxanthine phosphoribosyltransferase, secreted protein acidic and rich in cysteine, thioredoxin, or thioredoxin reductase.

4. The process of claim 1, wherein the at least one purification tag is chosen from fluorescent protein, His tag, poly(His) tag, FLAG tag, Halo tag, AcV5 tag, AU1 tag, AU5 tag, biotin carboxyl carrier protein, calmodulin binding protein, chitin binding domain, E tag, E2 tag, ECS tag, eXact tag, Glu-Glu tag, glutathione-S-transferase, HA tag, HSV tag, KT3 tag, maltose binding protein, MAP tag, Myc tag, NE tag, NusA tag, PDZ tag, S tag, S1 tag, SBP tag, Softag 1 tag, Softag 3 tag, Spot tag, Strep tag, SUMO tag, T7 tag, tandem affinity purification (TAP) tag, V5 tag, VSV-G tag, or Xa tag.

5. The process of claim 1, wherein the mammalian cell line is engineered to have at least one sequence encoding the purification tag integrated in-frame into a chromosomal sequence encoding the at least one host cell protein.

6. The process of claim 5, wherein the at least one sequence encoding the purification tag is located at the 5′ end and/or the 3′ end of the chromosomal sequence encoding the at least one host cell protein.

7. The process of claim 1, wherein the at least one sequence encoding the purification tag is integrated into the chromosomal sequence using a targeting endonuclease-mediated genome modification technique.

8. The process of claim 7, wherein the targeting endonuclease is a CRISPR ribonucleoprotein complex or a pair of zinc finger nucleases.

9. The process of claim 1, wherein the mammalian cell line is a human cell line chosen from a human embryonic kidney cell 293 (HEK293) cell line, a HT-1080 human connective tissue line, or a PER.C6 human embryonic retinal cell line.

10. The process of claim 1, wherein the mammalian host cell line a non-human cell line chosen from a Chinese hamster ovary (CHO) cell line, a baby hamster kidney (BHK) cell line, a NS0 mouse myeloma cell line, a Sp2/0 mouse myeloma cell line, a C127 mouse mammary gland cell line, or a Vero African green monkey kidney cell line.

11. The process of claim 1, wherein the mammalian cell line is a CHO cell line.

12. The process of claim 1, wherein the recombinant protein is chosen from an antibody, an antibody fragment, a vaccine, a growth factor, a cytokine, a hormone, or a clotting factor.

13. The process of claim 1, wherein the at least one tagged host cell protein is removed from the recombinant protein during the purification step by affinity chromatography, immunoaffinity chromatography, immunoprecipitation, or a combination thereof.

14. The process of claim 13, wherein the purification step comprises a clarification step prior to the removal the at least one tagged host cell protein.

15. The process of claim 14, wherein the purification step further comprises one or more chromatography steps.

16. The process of claim 15, wherein the level of residual host cell protein in the recombinant protein product is less than 100 ppm.

17-27 (canceled)
